(19) United States
(12) Patent Application Publication
(10) Pub. No.: US 2002/0128541 A1
(43) Pub. Date: Sep. 12, 2002

(54) Visual displaying device for virtual reality with a built-in biofeedback sensor

(76) Inventors: Sun-II Kim, Seoul (KR); Jan Han Lee, Anyang-si (KR); Dong Joo Kang, Seoul (KR); Dong-Pyo Jang, Sungnam-city (KR); In-Young Kim, Seoul (KR); Jae-Seok Kim, Seoul (KR); Back-Hwan Cho, Pusan (KR); Jeong-Hun Ku, Pusan (KR)

Correspondence Address: Welsh & Katz, Ltd., Thomas W. Tolpin, 22nd Floor, 120 South Riverside Plaza, Chicago, IL, 60606, US

(21) Appl. No.: 10/083,082

(22) Filed: Feb. 26, 2002

(30) Foreign Application Priority Data
Feb. 26, 2001 (KR) 10-2001-0009546

(51) Int. Cl. A61B 5/00; A61B 5/04

(52) U.S. Cl. 600/301; 600/509

(57) ABSTRACT

The present invention discloses a visual displaying device comprising a head mounted display, a biofeedback sensor, a head tracker, and three-dimensional sound generator for implementing virtual reality with biofeedback.

### FIELD OF THE INVENTION

The present invention relates to a visual displaying device for virtual reality, and more particularly to a device with biofeedback sensor.

### BACKGROUND OF THE INVENTION

The virtual reality (VR) is a novel technology that enables the user to enter a computer-generated world and interact with computer through vision, auditory sense, and sense of touch.

The virtual reality differs from the traditional displaying technique in a sense that it provides the user with the feeling of existence or the feeling of absorption as well as a variety of the computer graphic interface.

The virtual reality provides a paradigm where human being interacts with the computer, and consequently the user is no longer an observer that simply appreciates the computer image on the screen.

The user in virtual reality is absorbed in the activity in the three-dimensional virtual world that is generated by the computer.

The virtual environment is sometimes called in different terms such as virtual reality, remote reality, artificial world, cyber space, and multi-sense I/O (input and output).

As a displaying means for virtual reality, an HMD (head mounted display) and a shutter glass is widely used.

The shutter glass can provide a monitor-based three-dimensional image by shutting off the human eyes. Further, the HMD usually employed for VR is an essential device for providing the user with the feeling of absorption and existence.

Examples of patents and patent applications relating to head mounted displays are given in U.S. Pat. Nos. 4,695,129 and 5,900,849.

The conventional HMD, however, has a shortcoming in a sense that the HMD does not have a feedback unit monitoring the status of the user. Furthermore, the wearer of the conventional HMD should prepare an addition head tracker in order to locate the three-dimensional movement of the wearer's view.

### BRIEF SUMMARY OF THE INVENTION

In view of these problems, there is need in the art for devising a visual displaying device implementing both the virtual reality and the biofeedback.

Accordingly, it is an object of the present invention to provide a visual displaying device having a brain-wave sensing unit and a head tracker for implementing virtual reality.

Yet it is another object of the present invention to provide a visual displaying device implementing virtual reality by merging a biofeedback sensor, a head tracker, and a VR sound system in a single unit.

In accordance with a broad aspect of the present invention, provided is a visual displaying device having all the features of an HMD, a biofeedback sensor, a head tracker, and a VR sound system.

### BRIEF DESCRIPTION OF THE DRAWINGS

Further features of the present invention will become apparent from a description of a visual displaying device taken in conjunction with the accompanying drawings of the preferred embodiment of the invention, which, however, should not be taken to be limitative to the invention, but are for explanation and understanding only.

In the drawings:

FIG. 1 is a schematic diagram illustrating the structure of a visual displaying device in accordance with the present invention.

FIG. 2 is a schematic diagram illustrating an exemplary application of a visual displaying device in accordance with the present invention.

FIG. 3 is a schematic diagram illustrating a preferred embodiment of a shutter glass in accordance with the present invention.

### DETAILED DESCRIPTION OF THE INVENTION

Preferred embodiments in accordance with the present invention will be explained in detail with reference to the accompanying drawings.

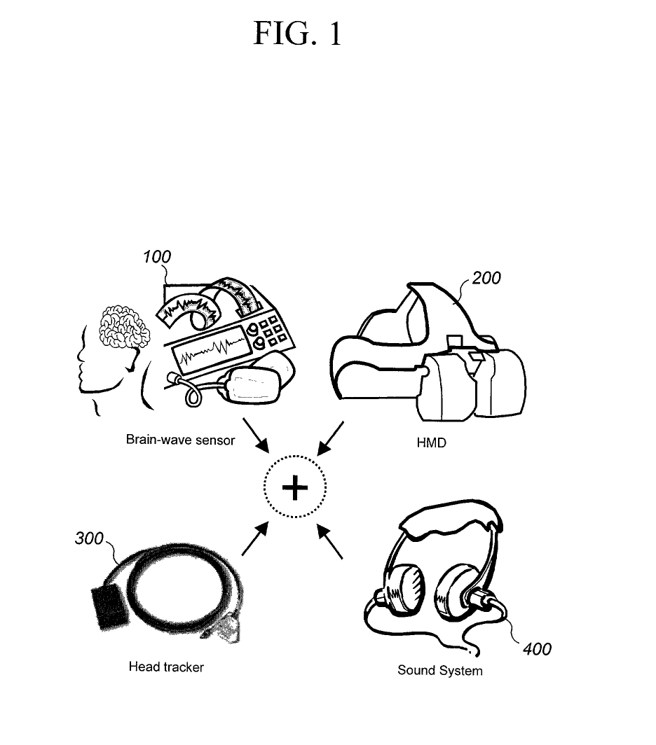

FIG. 1 is a schematic diagram illustrating the structure of a visual displaying device in accordance with the present invention.

Referring to FIG. 1, the present invention discloses a visual displaying device comprising a brain-wave sensing unit 100 for biofeedback.

The brain-wave sensing unit 100 in accordance with the present invention can include a brain-wave sensor detecting alpha-wave, beta-wave, theta-wave, and SRM-wave, and a signal transducer converting the above-mentioned brain waves into an electric signal for a feedback to a computer system.

The head tracker 300 in accordance with the present invention detects the 3-dimensional coordinates of the user's location in virtual reality. Further, the HMD 200 and a sound generator 400 for providing three-dimensional sound are also included.

The visual displaying device for virtual reality in accordance with the present invention comprises a helmet that is wearable by a user in virtual reality and a biofeedback sensor that is installed at the inner surface of said helmet and is in contact with the head of the helmet wearer for extracting brain-waves.

Furthermore, the visual displaying device further comprises an HMD attached at the front of said helmet, convertible upward and downward for in sue and stand-by and a sound generator proving a sound in accordance with visual VR contents through said HMD, installed at both the inner sides of said helmet in such a way that the speaker of said sound generator is in contact with the ears of the helmet wearer.

The biofeedback sensing unit in accordance with the present invention comprises a brain-wave sensor detecting either one or a combination from the group of alpha-wave, beta-wave, theta-wave, and SRM-wave and a transducer converting said brain-wave into an electric signal for biofeedback to a computer.

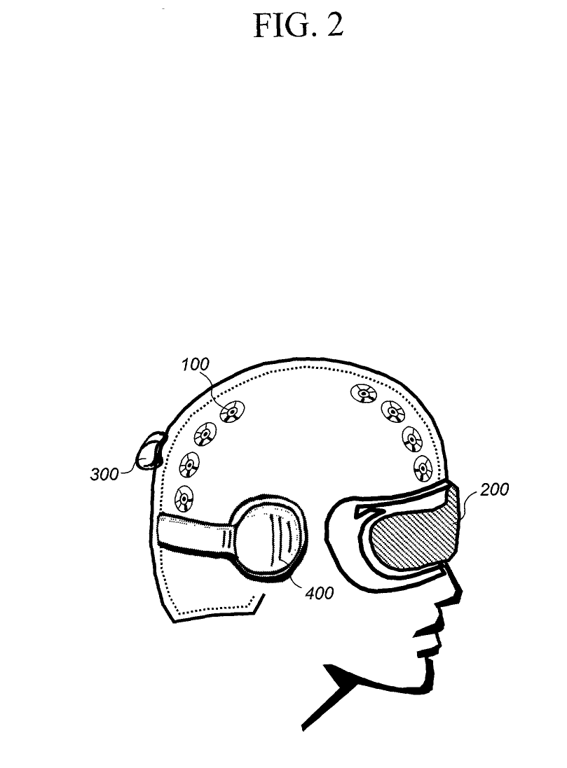

FIG. 2 is a schematic diagram illustrating an exemplary application of a visual displaying device in accordance with the present invention. Referring to FIG. 2, a helmet-type device is disclosed in which a brain-wave sensor 100 as well as a sound generator is prepared.

The HMD 200 in accordance with the present invention is installed at the front and is convertible upward and downward like a motorcycle helmet.

Furthermore, a head tracker 300 can be installed at the back of the visual displaying device for tacking the wearer's movement in virtual reality.

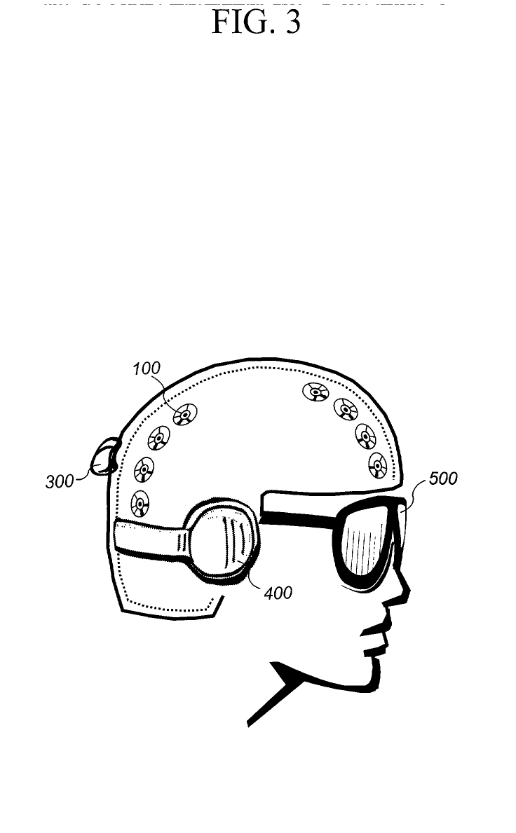

FIG. 3 is a schematic diagram illustrating a preferred embodiment of a shutter glass in accordance with the present invention. Referring to FIG. 3, the preferred embodiment comprises a helmet in which a brain-wave sensor is prepared. The sound generator is also prepared on both the inner sides of the helmet.

The shutter glass 500 in accordance with the present invention can be installed at the front and can be designed such that it is convertible.

More preferably, a head tracker 300 can be implemented inside the helmet for tracking the three-dimensional the wearer's movement in the virtual reality through the shutter glass 500.

Although the invention has been illustrated and described with respect to exemplary embodiments thereof, it should be understood by those skilled in the art that various other changes, omissions and additions may be made therein and thereto, without departing from the spirit and scope of the present invention.

Therefore, the present invention should not be understood as limited to the specific embodiment set forth above but to include all possible embodiments which can be embodies within a scope encompassed and equivalents thereof with respect to the feature set forth in the appended claims.

What is claimed is:

1. A visual displaying device for virtual reality, comprising:
a helmet that is wearable by a user in virtual reality;
a biofeedback sensor that is installed at the inner surface of said helmet and is in contact with the head of the helmet wearer for extracting brain-waves;
an HMD attached at the front of said helmet, convertible upward and downward for in sue and stand-by; and
a sound generator proving a sound in accordance with visual VR contents through said HMD, installed at both the inner sides of said helmet in such a way that the speaker of said sound generator is in contact with the ears of the helmet wearer.

2. The visual displaying device as set forth in claim 1 wherein said biofeedback sensing unit comprises
a brain-wave sensor detecting either one or a combination from the group of alpha-wave, beta-wave, theta-wave, and SRM-wave; and
a transducer converting said brain-wave into an electric signal for biofeedback to a computer.

3. The visual displaying device as set forth in claim 1 wherein said HMD is replaced by a shutter glass.
